(19) United States
(12) Patent Application Publication
(10) Pub. No.: US 2005/0096253 A1
(43) Pub. Date: May 5, 2005

(54) Utilization of inhibitors of rho-kinases for stimulating nerve growth, inhibiting scar tissue formation and/or reducing a secondary lesion

(76) Inventors: Philippe P. Monnier, Tubingen (DE); Bernd Stahl, Merburg (DE); Bernhard K. Muller, Neustadt (DE); Alexander Domling, Munchen (DE); Werner Schiebler, Wiesbaden (DE)

Correspondence Address: PALMER & DODGE, LLP;PAULA CAMPBELL EVANS, 111 HUNTINGTON AVENUE, BOSTON, MA, 02199, US

(21) Appl. No.: 10/494,093

(22) PCT Filed: Oct. 31, 2002
(86) PCT No.: PCT/EP02/12223

(30) Foreign Application Priority Data
Nov. 2, 2001 (DE) 101 53 605.4

(51) Int. Cl. A61K 38/00

(52) U.S. Cl. 514/2

(57) ABSTRACT

The present invention relates to the use of inhibitors of rho-kinases in the in vivo stimulation of nerve growth, in the in vivo inhibition of scar tissue formation and/or in the in vivo reduction of secondary damage.

The present invention relates to the use of inhibitors of rho-kinases in the in vivo stimulation of nerve growth, in the in vivo inhibition of scar tissue formation and/or in the in vivo reduction of secondary damage.

In vertebrates, the spinal cord and the brain form the central nervous system (CNS). The spinal cord runs in the longitudinal direction of the body and is surrounded by the spinal canal. In humans, it can be sub-divided into eight cervical, twelve thoracic, five lumbar, five sacral and one or two coccygeal segments. The central grey matter with its lateral bulges (anterior and posterior horns) is formed by the nerve cell bodies, and the peripheral white matter is formed by the medullated nerve fibre bundles. Afferent (ascending, sensory) and efferent (descending, effectory) conduction tracts extend in the white matter. The descending tracts of the spinal cord are sub-divided into pyramidal tracts (voluntary movements) and extrapyramidal tracts (involuntary movements; distribution of muscle tonus). The pyramidal fibres extend, for the most part having crossed over in the lateral pyramidal tract of the opposite side and for the lesser part not having crossed over in the anterior pyramidal tract, to anterior horn cells and posterior horn cells of the various spinal cord segments.

Injury to the spinal cord, for example as a result of an accident, results in lasting interruption of the conduction function of the affected nerve fibres. Paralysis resulting from an accident is caused by lasting interruption of the conduction function of the affected nerve fibres. Paralysis resulting from complete failure of at least one segment is referred to as paraplegia or quadriplegia. The consequence is the loss of sensory (for example, temperature, pain and pressure sensations), motor (voluntary and involuntary movements) and autonomic functions (for example bladder and bowel function) for all areas below the affected segment. Because of the poor regenerative capabilities of the nerve fibres, the paralysis of voluntary motor function and the complete sensory loss will be lasting.

However, there are also neurological and neurodegenerative diseases of the peripheral and central nervous system in which nerve cells are destroyed. These are, for example, Alzheimer's disease, Parkinson's disease, multiple sclerosis and similar diseases involving nerve fibre loss and demyelination, as well as amyotrophic lateral sclerosis and other motor neuron diseases, ischaemia, stroke, epilepsy, Huntington's disease, AIDS dementia complex and prion diseases.

Research is therefore aimed at bringing about regeneration of the nerve axons in the case of lesions of the spinal cord and, in the case of other diseases of the peripheral and central nervous system, at stimulating nerve growth. An injury to the brain or spinal cord of humans leads, within days and weeks, to the formation of massive scar tissue, which constitutes an impenetrable barrier to regenerating nerve fibres. The basic element of that scar tissue is a structural matrix of collagenous fibres in which nerve fibre growth inhibitors are embedded. Those regeneration inhibitors include proteins (for example, RGM or repulsive guidance molecule) and proteoglycans, that is to say proteins having a high sugar or carbohydrate content. For that reason, slowing down or preventing scar formation and stimulating nerve fibre growth are fundamental therapeutic aims in neurodegenerative treatment concepts.

The problem of the present invention is accordingly to stimulate nerve growth in vivo and, especially, to inhibit scar tissue formation in vivo.

In accordance with the invention it has been found that the above-mentioned proteins and proteoglycans act by means of activation of rho-kinases, causing inhibition of nerve fibre regeneration as a result.

By employing or using specific inhibitors of rho-kinases it is possible, in accordance with the invention, to neutralise the inhibitory action of those regeneration inhibitors. As a consequence of that neutralisation, strong growth of new nerve fibres and, associated therewith, regeneration of damaged, interrupted neuronal connections are brought about.

The present invention accordingly relates to the use of inhibitors of rho-kinases, especially of human rho-kinases, (especially of the compounds described in the Examples), in the in vivo stimulation of nerve growth, especially of mammals, in the in vivo inhibition of scar tissue formation, especially of mammals, especially following damage to the brain, spinal cord or other nerves, especially of humans, and/or in the in vivo reduction of secondary damage, especially of mammals, especially following damage to the brain, spinal cord or other nerves, especially of humans.

### EXAMPLES

Examples of known rho-kinase inhibitors are, for example, the compounds, described in EP 0 956 865 and U.S. Pat. No. 4,997,834, of the general formula (I): 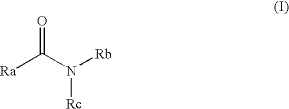
wherein

Ra is a group of formula (a), (b) or (c): 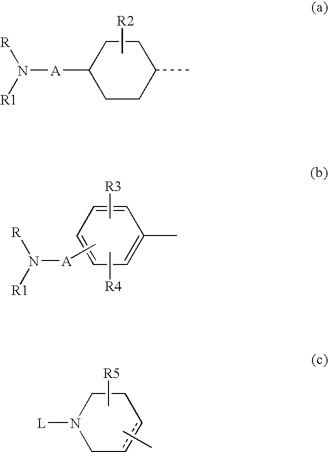
wherein

R and R1 are each independently of the other a hydrogen atom, an alkyl, heteroalkyl, aryl, heteroaryl, cycloalkyl, heterocycloalkyl, aralkyl or heteroaralkyl radical or together are part of a heterocycloalkyl ring;

R2 is a hydrogen atom or an alkyl radical;

R3 and R4 are each independently of the other a hydrogen atom, a halogen atom, a hydroxy, amino, nitro or thiol group, an alkyl, heteroalkyl, aryl, heteroaryl, cycloalkyl, heterocycloalkyl, aralkyl or heteroaralkyl radical;

A is a group of formula —(CH₂)ₗ—(CR6R7)ₘ-(CH₂)ₙ- wherein R6 and R7 are each independently of the other a hydrogen atom, an alkyl, heteroalkyl or aralkyl radical or together are part of a cycloalkyl ring and l, m and n are each independently of the others 0 or a whole number from 1 to 3;

L is a hydrogen atom, an alkyl, heteroalkyl, aryl, heteroaryl, cycloalkyl, heterocycloalkyl, aralkyl or heteroaralkyl radical;

R5 is a hydrogen atom, a hydroxy, alkoxy, alkoxycarbonyloxy, alkylcarbonyloxy or aralkyloxycarbonyloxy group;

Rb is a hydrogen atom, an alkyl, aralkyl, aminoalkyl or mono- or di-alkylaminoalkyl group, and

Rc is an optionally substituted heterocycloalkyl radical containing at least one nitrogen atom;

or a pharmacologically acceptable salt, solvate, hydrate or a pharmacologically acceptable formulation thereof.

Further examples of known rho-kinase inhibitors are the compounds, described in EP 0 956 865 and U.S. Pat. No. 4,678,783, of the general formula (II): 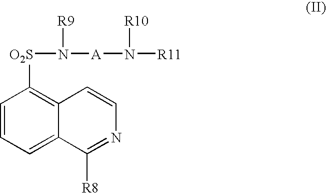
wherein

R8 is a hydrogen atom, a halogen atom or a hydroxy group;

R9 and R10 are each independently of the other a hydrogen atom or an alkyl group or together with the group —N-A-N— are part of a heterocycloalkyl ring;

R11 is a hydrogen atom or an alkyl or heteroalkyl group, and

A is an alkylene group containing from 2 to 6 carbon atoms;

or a pharmacologically acceptable salt, solvate, hydrate or a pharmacologically acceptable formulation thereof.

Further examples of known rho-kinase inhibitors are the compounds, described in U.S. Pat. No. 6,153,608, of the general formula (III): 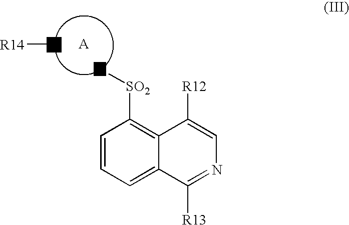
wherein

R12 is a halogen atom, an alkyl or heteroalkyl radical;

R13 is a hydrogen atom, a hydroxy group or a halogen atom;

R14 is a hydrogen atom, an alkyl or heteroalkyl radical, and

A is a 5- to 11-membered heterocycloalkyl ring, which may additionally comprise an alkylene ring, for example bonded to two hydrocarbon atoms of the heterocycloalkyl ring;

or a pharmacologically acceptable salt, solvate, hydrate or a pharmacologically acceptable formulation thereof.

Further examples of known rho-kinase inhibitors are the compounds, described in WO0156988, of the general formula Het-X-Q-Z (IV) wherein

Het is a mono- or bi-cyclic heterocycloalkyl group containing at least one nitrogen (for example, pyridyl, phthalimido, quinolyl, indazolyl);

X is an oxygen atom or a group of formula —NH—CO—NH—, —NH—CO—, —NR15-, R15 being a hydrogen atom, an alkyl or heteroalkyl radical;

Q is a direct bond, an alkylene, heteroalkylene, cycloalkylene or heterocycloalkylene group, and

Z is an aryl, aralkyl, heteroaryl, heteroarylalkyl, cycloalkyl or heterocycloalkyl group;

or a pharmacologically acceptable salt, solvate, hydrate or a pharmacologically acceptable formulation thereof.

The expression “alkyl” refers to a saturated or at least partially unsaturated (for example, alkenyl, alkynyl), straight-chain or branched hydrocarbon group, containing 1 or from 2 to 20 carbon atoms, preferably 1 or from 2 to 12 carbon atoms, especially 1 or from 2 to 6 carbon atoms, for example the methyl, ethyl, isopropyl, iso-butyl, tert-butyl, n-hexyl, 2,2-dimethylbutyl, n-octyl, allyl, isoprenyl or hex-2-enyl group.

The term “heteroalkyl” refers to an alkyl group in which one or more (preferably 1, 2 or 3,) carbon atoms have been replaced by an oxygen, nitrogen, phosphorus or sulfur atom (preferably oxygen or nitrogen), for example an alkyloxy group such as, for example, methoxy or ethoxy, or a methoxymethyl, nitrile, methylcarboxyalkyl ester, carboxyalkyl ester or 2,3-dioxyethyl group. The term “heteroalkyl” refers furthermore to a carboxylic acid or to a group derived from a carboxylic acid such as, for example, acyl, acyloxy, carboxyalkyl, carboxyalkyl ester, for example methylcarboxyalkyl ester, carboxyalkylamide, alkoxycarbonyl or alkoxycarbonyloxy.

The terms “cycloalkyl” or “cyclo-” refer to a saturated or partially unsaturated cyclic group comprising one or more rings forming a structure containing from 3 to 14 carbon atoms, preferably from 3 to 10 carbon atoms, for example the cyclopropyl, cyclohexyl, Tetralin or cyclohex-2-enyl group.

The expression “heterocycloalkyl” or “heterocyclo-” refers to a cycloalkyl group as defined above, in which one or more (preferably 1, 2 or 3) carbon atoms have been replaced by an oxygen, nitrogen, phosphorus or sulfur atom and can denote, for example, the piperidine, morpholine, N-methylpiperazine or N-phenylpiperazine group.

The expression “aryl” or “ar” refers to an aromatic group comprising one or more rings and being formed by a structure containing from 5 to 14 carbon atoms, preferably 5 or from 6 to 10 carbon atoms, for example a phenyl, naphthyl, 2-, 3- or 4-methoxyphenyl, 2-, 3- or 4-ethoxyphenyl, 4-carboxyphenylalkyl or 4-hydroxyphenyl group.

The expression “heteroaryl” refers to an aryl group in which one or more (preferably 1, 2 or 3) carbon atoms have been replaced by an oxygen, nitrogen, phosphorus or sulfur atom, for example the 4-pyridyl, 2-imidazolyl, 3-pyrazolyl and isoquinolyl group.

The expressions “aralkyl” and “heteroaralkyl” refer to groups including, in accordance with the above definitions, both aryl and heteroaryl and also alkyl and/or heteroalkyl and/or cycloalkyl and/or heterocycloalkyl ring systems, for example the tetrahydroisoquinolyl, benzyl, 2- or 3-ethyl-indolyl or 4-methylpyridino group.

The expressions “alkyl”, “heteroalkyl”, “cycloalkyl”, “heterocycloalkyl”, “aryl”, “heteroaryl”, “aralkyl” and “heteroaralkyl” also refer to groups in which one or more hydrogen atoms of such groups have been replaced by fluorine, chlorine, bromine or iodine atoms or by OH, SH, NH₂ or NO₂ groups. Those expressions furthermore refer to groups which are substituted by unsubstituted alkyl, heteroalkyl, cycloalkyl, heterocycloalkyl, aryl, heteroaryl, aralkyl or heteroaralkyl groups.

The above-described compounds of the general formulae (I), (II), (III) and (IV) may, by virtue of their substitution, contain one or more chiral centres. The present invention accordingly includes all pure enantiomers and all pure diastereomers, and also mixtures thereof in any mixing ratio.

Especially preferred rho-kinase inhibitors are compounds of formulae (V), (VI) and (VII):

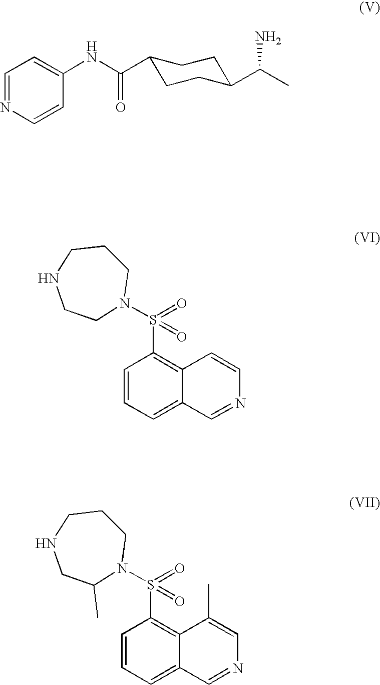

1. A method for stimulating nerve growth in vivo comprising administering an inhibitors of a rho-kinase.

2. A method for inhibiting scar tissue formation by administering an inhibitor of a rho-kinase.

3. A method for inhibiting reduction of secondary damage by administering an inhibitor of a rho-kinase.

4. A method for neutralizing regeneration inhibitors of scar tissue by administering an inhibitor of a rho-kinase.

5. A method for neutralizing regeneration inhibitors of the myelin sheath and oligodendrocytes by administering an inhibitor of a rho-kinase.

6. A method for neutralizing regeneration inhibitors on the surface of astrocytes, macrophages and microglial cells by administering an inhibitor of a rho-kinase.

7. A method for neutralizing regeneration inhibitors on the surface of lesion-associated cells of the immune system by administering an inhibitor of a rho-kinase.

8. A method for treating neuron damage by administering an inhibitor of a rho-kinase.

9. A method for treating acute injuries to the brain and spinal cord by administering an inhibitor of a rho-kinase.

10. A method for treating chronic damage to the brain and spinal cord by administering an inhibitor of a rho-kinase.

11. A method for treating neurological and neurodegenerative diseases of the central nervous system by administering an inhibitor of a rho-kinase.

12. The method of claim 11 wherein treatment of the neurological and neurodegenerative disease comprises regenerative treatment of Alzheimer's disease, Parkinson's disease, multiple sclerosis, a diseases associated with nerve fibre loss and demyelination, amyotrophic lateral sclerosis or a motor neuron diseases.

13. The method of claim 11 wherein treatment of the neurological and neurodegenerative disease comprises regenerative treatment of ischaemia, stroke, epilepsy, Huntington's disease, AIDS dementia complex or a prion diseases.

14. A method for treating neurological and neurodegenerative diseases of the peripheral nervous system by administering an inhibitor of a rho-kinase.

15. The method according to claim 14 wherein the disease comprises a lesions of peripheral a nerves.

16. The method according to claim 14 wherein the disease comprises paralysis caused by injuries to peripheral nerves.

17. A method for treating deficits of memory and of recall by increasing cerebral blood flow, the method comprising administering an inhibitor of a rho-kinase.

18. The methods of claims 1-11, 14 and 17 wherein the rho-kinase inhibitor comprises a compound of formula (V), (VI) or (VII): [chemical structure]
